(19) United States
(12) Patent Application Publication
(10) Pub. No.: US 2012/0035531 A1
(43) Pub. Date: Feb. 9, 2012

(54) ON-DEMAND AND REVERSIBLE DRUG RELEASE BY EXTERNAL CUE

(75) Inventors: Xuanhe Zhao, Cambridge, MA (US); Nathaniel D. Huebsch, Colma, CA (US); David J. Mooney, Sudbury, MA (US); Zhigang Suo, Lexington, MA (US)

(73) Assignee: PRESIDENT AND FELLOWS OF HARVARD COLLEGE, Cambridge, MA (US)

(21) Appl. No.: 13/147,870

(22) PCT Filed: Feb. 1, 2010
(86) PCT No.: PCT/US10/22769

§ 371 Date: Oct. 17, 2011

Related U.S. Application Data
(62) Division of application No. 61/151,277, filed on Feb. 10, 2009.

(51) Int. Cl. A61M 37/00

(52) U.S. Cl. 604/22

(57) ABSTRACT

The invention provides a method to release drugs from a polymer matrix upon demand without degrading the matrix. By applying ultrasound to a self-healable polymer matrix in physiological environment, compounds of both low-molecular and high-molecular weights encapsulated in the matrix are delivered at controlled rates, while the integrity and stiffness of the matrix are unaffected.

### RELATED APPLICATIONS

This application claims benefit under 35 U.S.C. §119(e) of the U.S. Provisional Application No. 61/151,277, filed Feb. 10, 2009, the content of which are incorporated herein by reference in its entirety.

### GOVERNMENT SUPPORT

This invention was made with government support under grant no. R37 DE013033 awarded by the National Institutes of Health. The government has certain rights in this invention.

### FIELD OF THE INVENTION

The invention relates to controlled release of drugs and biologics in a controlled manner at specific locations in the body in response to an external stimulus.

### BACKGROUND OF THE INVENTION

Over the recent decades, there have been many advances in the development of biocompatible polymer systems for drug delivery. Generally, drugs of low-molecular weight or high-molecular weight are encapsulated into a cross-linked polymer matrix and are subsequently released in a physiological environment due to physical or chemical changes, which are typically irreversible, in the polymer. Examples of such changes include hydration and swelling state, collapse, rupture and degradation (Langer 1990; Langer 1998; Uhrich 1999). This requirement for either permanent, irreversible change or significant temporary changes in the macroscale properties of the polymer is problematic.

For example, some drugs may bind, through various interactions, with the polymer matrix during encapsulation, and become released as the cross-linked polymer matrix degrades. (Bouhadir 2001). However, drugs released in this manner may still be bound to polymer released from the matrix, and therefore have limited bioactivity. In other cases, degradation products or environment changes in pH and osmolality induced by polymer degradation may affect activity or the structural integrity of drug compounds. As another example, drug-releasing polymer matrices may be used as bulking agents or wound dressing (Thornton 2004), and both degradation or significant changes in physical properties (e.g. swelling), will limit their function in this regard.

Recently, efforts have also been made to release drugs from polymer matrix upon demand and at controlled rates. Much of this work has been on the use of stimulus that could be applied non-invasively in the clinic (e.g. magnetic fields, Cheng 2006). Mechanical stresses have also been applied on polymer matrix containing drugs to stimulate their delivery. (U.S. Pat No. 6,748,954) The release rate is increased with the application of mechanical stresses, but the increase cannot be accurately controlled.

One form of mechanical stress, low-frequency ultrasound, has been applied on polymer matrices. The ultrasound energy accelerates degradation of the polymer, by creating cavitations, and exerting mechanical stress, leading to drug release. (U.S. Pat No. 4,657,543) However, because the degradation of polymer is difficult to control precisely with ultrasound, the enhanced release rate of drugs gives irregular patterns as shown in FIG. 2 of U.S. Pat No. 4,657,543. In addition, the degraded products of the polymer may introduce some of the problems described above.

It would therefore be advantageous to provide a polymer matrix capable of on-demand and reversible drug release at controlled rates, without degrading the polymer matrix or decreasing its mechanical strength.

### SUMMARY OF THE INVENTION

In one aspect, the invention provides a method for releasing a bioactive agent on demand in response to an external cue, the method comprising providing a physiologically acceptable self-healing polymer matrix comprising the bioactive agent and inducing cavitation in the polymer matrix via ultrasound to release the drug.

### BRIEF DESCRIPTION OF THE DRAWINGS

FIGS. 1A-1F show the release profiles of Mitoxantrone from Alginate hydrogel with 5 min of 1 W ultrasound each hour. FIGS. 1A and 1B show the power profiles of ultrasound in the experimental (FIG. 1A) and control (FIG. 1B) groups of alginate hydrogels. FIGS. 1C and 1D show the corresponding release rates. FIGS. 1E and 1F show the corresponding cumulative release.

FIG. 2 shows the bioactivity of Mitoxantrone released from Alginate hydrogel with ultrasound, as determined by the efficacy of the drug in killing MCF7 breast cancer cells in vitro.

FIGS. 3A-3B show the release rates of plasmid DNA from Alginate hydrogel with 15 min of 1 W ultrasound each day. FIG. 3A shows the power profile of the ultrasound. FIG. 3B shows the release rate of plasmid DNA.

FIGS. 4A-4B show effect of ultrasound irradiation on hydrogels in media with normal Ca²⁺ and Mg²⁺ concentration, with 5 times of normal Ca²⁺ and Mg²⁺ concentration, and PBS without Ca²⁺ and Mg²⁺. FIG. 4A shows the variation of modulus, and FIG. 4B shows the dry polymer weight of irradiated hydrogels.

FIG. 5 shows a schematic representation of Mitoxantrone forming ionic complex with sugar residues of alginate polymer backbone.

### DETAILED DESCRIPTION OF THE INVENTION

In one aspect, the invention provides a method for releasing a bioactive agent on demand in response to an external cue, the method comprising providing a physiologically acceptable self-healing polymer matrix comprising the bioactive agent and inducing cavitation in the polymer matrix via ultrasound to release the drug. As used herein, the term “on-demand” refers to the operator control over the release of bioactive agent from the composition.

The method can be used for releasing a bioactive agent on demand in a specified location in a patient in response to an external cue. This is accomplished by providing to a location within the patient a physiologically acceptable self-healing polymer matrix comprising the bioactive agent and inducing cavitation in the polymer matrix via ultrasound to release the drug.

The method for drug delivery provided by the invention utilizes polymer matrices with reversible cross-links, so that the polymer matrices can maintain their integrity and stiffness in a physiological environment while releasing drugs in response to ultrasound. The ultrasound shock introduces cavitations into the polymer matrix and exerts mechanical pressures on it. As the drugs are released through the cavities, the reversibly physical cross-links in the cavities reform under physiological ionic conditions. This class of polymers is, therefore, self-healable in physiological environment.

As used herein, the term “self-healing” refers to ability of the polymer matrix to substantially return to an initial state or condition prior to exposure to an external stimulus and/or the ability to resist the formation of macroscopic or visual irregularities and/or defects that persist for a significant time after the exposure to external stimulus is terminated.

It is to be understood that external stimulus can be applied by providing a stimulus that is not present, by holding back a stimulus that is already present, or by changing the amount of a stimulus that is already present.

The present invention utilizes a type of polymers that are self-healable under physiological conditions and/or environment. The polymer matrix needs to be susceptible to low frequency ultrasound to create cavitations in it with ultrasound, in order to enhance bioactive agent release. Furthermore, the polymer matrix needs to re-heal the cavities in order to maintain its integrity and mechanical stiffness. Considering these requirements, any polymeric material, which can be reversibly cross-linked, can be used as the matrices of this invention.

The polymeric matrix material can be natural or synthetically derived. The matrix can comprise materials of synthetic or natural origin (e.g., biopolymers) or a mixture thereof. In some embodiments, the matrix material is reversibly cross-linked by physical and/or chemical interactions. In some embodiments, the matrix is not biodegradable. In some embodiments, the matrix is biocompatible. Polymer matrices can be prepared using methods known in the art and easily adapted by one of skill in the art. As used herein, the term “reversibly cross-linked” refers to cross-linked matrix where the cross-links can be broken under application of an external cue and then reform when the external cue is removed. In some embodiments, the matrix is reversibly cross-linked under physiological conditions.

Suitable matrices include polymers, copolymers, and blockpolymers based on monomers containing ionizable groups or polymerizable double bonds. Exemplary monomers include, but are not limited to, acrylic acid, methyl methacrylate, methyl acrylic acid, ethyl acrylate, vinyl sulfonic acid, styrene, styrene sulfonic acid (e.g., p-styrene sulfonic acid), maleic acid, butenoic acid, vinyl phosphate, vinyl phosphonate, ethylene, propylene, styrene, vinyl methyl ether, vinyl acetate, vinyl alcohol, acrylonitrile, acrylamide, N-(C₁-C₆ alkyl) acrylamide (such as N-isopropylacrylamide, N-t-butylacrylamide), and the like. Polymer matrices are made by homopolymerizing or copolymerizing any of the foregoing monomers. Other suitable polymer matrix materials can include, alginate, chitosan, collagen, gelatin, hyaluronate, fibrin, agarose, and derivatives thereof. The matrix can be a copolymer as described above into which has been incorporated as one comonomeric component a ligand that connects to, complexes or physically entraps the desired bioactive agent.

In some embodiments, matrix comprises a polymer selected from the group consisting of polyanhydrides, polyhydroxybutyric acid, polyorthoesters, polysiloxanes, polycaprolactone, poly(lactic-co-glycolic acid), poly(lactic acid), poly(glycolic acid), and copolymers prepared from the monomers of these polymers.

Suitable polymers which can be used in the present invention include but are not limited to one or a mixture of polymers selected from the group consisting of glycosaminoglycan, silk, fibrin, MATRIGEL®, poly-ethyleneglycol (PEG), polyhydroxy ethyl methacrylate, polyvinyl alcohol, polyacrylamide, poly (N-vinyl pyrolidone), poly glycolic acid (PGA), poly lactic-co-glycolic acid (PLGA), poly e-carpolactone (PCL), polyethylene oxide, poly propylene fumarate (PPF), poly acrylic acid (PAA), hydrolysed polyacrylonitrile, polymethacrylic acid, polyethylene amine, alginic acid, pectinic acid, carboxy methyl cellulose, hyaluronic acid, heparin, heparin sulfate, chitosan, carboxymethyl chitosan, chitin, pullulan, gellan, xanthan, collagen, gelatin, carboxymethyl starch, carboxymethyl dextran, chondroitin sulfate, cationic guar, cationic starch as well as salts and esters thereof. Polymers listed above which are not ionically cross-linkable are used in blends with polymers which are ionically cross-linkable.

Other preferred polymers include esters of alginic, pectinic or hyaluronic acid and C2 to C4 polyalkylene glycols, e.g. propylene glycol, as well as blends containing 1 to 99 wt % of alginic, pectinic or hyaluronic acid with 99 to 1 wt % polyacrylic acid, polymethacrylic acid or polyvinylalcohol. Preferred blends comprise alginic acid and polyvinylalcohol. Examples of mixtures include but are not limited to a blend of polyvinyl alcohol (PVA) and sodium alginate and propyleneglycol alginate.

In some embodiments, the polymer matrix is an alginate or alginate derivative. In some preferred embodiments, the polymer matrix is an alginate or alginate derivative in the form of a three dimensional hydrogel. As used herein, the term “alginate” refers to any number of derivatives of alginic acid (e.g., calcium, sodium or potassium salts, or propylene glycol alginate). Alginate can be reversibly cross-linked by divalent ions available in physiological environment such as Ca²⁺, Mg²⁺, Ba²⁺, and Sr²⁺. See for example, PCT/US97/16890, contents of which are herein incorporated by reference in its entirety. Suitable alginate polymers can have molecular weight from 5,000 to 500,000 Daltons.

The polymer matrix can be cross-linked to let it take a physically stable form when hydrated or dehydrated. Suitable cross-linking can be provided by incorporating about 0.5 wt. % to about 1.5% wt. % of a cross-linking agent into the polymer matrix. Cross-linking can also be provided by incorporating about 0.01 mol % to about 15 mol % of the cross-linking agent in the polymer matrix.

Suitable cross-linking agents include compounds whose molecule has a plurality of reactive groups. Such molecular cross-linking agents may be N,N′-methylene-bis acrylamide or divinylbenzene (DVB), ethylene glycol dimethacrylate, divinyl ketone, vinyl methacrylate and divinyl oxalate. Ionic cross-linkage which uses ions such as metallic ions may also be employed. Cross-linkage using electromagnetic waves such as gamma rays is also possible. Cross-linking can also be based on electrostatic interactions (e.g., ionic interactions), hydrogen boding, hydrophobic interactions or (micro)crystal formation.

Ionically cross-linkable polymers can be anionic or cationic in nature and include but not limited to carboxylic, sulfate, hydroxyl and amine functionalized polymers. The cross-linking ions used to cross-link the polymers can be anions or cations depending on whether the polymer is anionically or cationically cross-linkable. Appropriate cross-linking ions include but not limited to cations selected from the group consisting of calcium, magnesium, barium, strontium, boron, beryllium, aluminum, iron, copper, cobalt, lead and silver ions. Anions can be selected from but not limited to the group consisting of phosphate, citrate, borate, succinate, maleate, adipate and oxalate ions. More broadly, the anions are derived from polybasic organic or inorganic acids. Preferred cross-linking cations are calcium, iron, and barium ions. The most preferred cross-linking cations are calcium, magnesium and barium ions. The most preferred cross-linking anion is phosphate. Cross-linking can be carried out by contacting the polymers with a nebulized droplet containing dissolved ions. One of ordinary skill in the art will be able to select appropriate cross-linking agent for the respective hydrogel. For example, the gelation of collagen or alginate occurs in the presence of ionic cross-linker or divalent cations such as Ca²⁺, Ba²⁺, Mg²⁺ and Sr²⁺.

In some embodiments, the polymer matrix is reversibly cross-linked by divalent cations. The divalent concentration capable of reversibly cross-linking the polymer matrix can range from about 0.001 mM to about 10 mM. Preferably, divalent concentration ranges from about 0.1 mM to 5 mM. In some embodiments, concentration of divalent cation is from about 0.01 mM to about 10 mM. In some embodiments, the divalent cation is selected from the group consisting of Ca²⁺, Ba²⁺, Mg²⁺, Sr²⁺, and combinations thereof.

In some embodiments, the polymer matrix can be reversibly cross-linked under physiological conditions. As used herein the term “physiological condition” refers to temperature, pH, ions, ionic strength, viscosity, and like biochemical parameters which exist extracellularly or intracellularly in an organism. In some embodiments, the physiological condition refers to conditions found in serum and/or blood of an organism. In some embodiments, the physiological condition refers conditions found in a cell in an organism.

Particular in vitro conditions to mimic physiological conditions can be selected by the practitioner according to conventional methods. For general guidance, the following buffered aqueous conditions can be applicable: 10-250 mM NaCl, 5-50 mM Tris HCl, pH 5-8, with optional addition of divalent cation(s) and/or metal chelators and/or nonionic detergents and/or membrane fractions and/or antifoam agents and/or scintillants. In general, in vitro conditions that mimic physiological conditions comprise 50-200 mM NaCl or KCl, pH 6.5-8.5, 20-45° C., and 0.001-10 mM divalent cation (e.g., Mg²⁺, Ca²⁺); preferably about 150 mM NaCl or KCl, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v).

The polymer matrix can be a swellable gel or a non swellable gel. Swellable gels can include hydrogels and organogels. The term “hydrogel” indicates a cross-linked, water insoluble, water containing material. Hydrogels have many desirable properties for biomedical applications. For example, they can be made nontoxic and compatible with tissue, and they are usually highly permeable to water, ions and small molecules.

The preferred hydrogels include collagen and gelatin, hyaluronate, fibrin, alginate, agarose, chitosan, poly(acrylic acid), poly(ethylene oxide), poly(vinyl alcohol), polyphosphazene, and polypeptides. Polymer concentration in the gel can range from 0.1% (w/w) to 40% (w/w). Preferably, polymer concentration in the gel is from 0.5-3%.

In some embodiments, polymer matrix has an elastic modulus in the range between 10⁻³ and 10³ kPa. As used herein, the term “elastic modulus” refers to an object or substance's tendency to be deformed elastically (i.e., non-permanently) when a force is applied to it. Generally, the elastic modulus of an object is defined as the slope of its stress-strain curve in the elastic deformation region. Specifying how stress and strain are to be measured, including directions, allows for many types of elastic moduli to be defined. Young's modulus (E) describes tensile elasticity, or the tendency of an object to deform along an axis when opposing forces are applied along that axis; it is defined as the ratio of tensile stress to tensile strain. It is often referred to simply as the elastic modulus. The shear modulus or modulus of rigidity (G or μ) describes an object's tendency to shear (the deformation of shape at constant volume) when acted upon by opposing forces; it is defined as shear stress over shear strain. The shear modulus is part of the derivation of viscosity. The bulk modulus (K) describes volumetric elasticity, or the tendency of an object to deform in all directions when uniformly loaded in all directions; it is defined as volumetric stress over volumetric strain, and is the inverse of compressibility. The bulk modulus is an extension of Young's modulus to three dimensions. Three other elastic moduli are Poisson's ratio, Lamé's first parameter, and P-wave modulus.

In some embodiments, the polymer matrix maintains physical integrity and mechanical stiffness that is within 24%, 10%, 5%, 2% or less of their initial values after repeated (e.g., 1, 2, 3, 4, 5, 6, 7, 8, 9, 10, 15, 20, 25, 50, 75, 100 or more) applications of ultrasound. As used herein, the term “physical integrity” refers to porosity, pore size, pore connectivity, specific volume, and combinations thereof, of the polymer matrix.

As used herein, “bioactive agents” or “bioactive materials” refer to naturally occurring biological materials, for example, extracellular matrix materials such as fibronectin, vitronection, and laminin; cytokins; and growth factors and differentiation factors. “Bioactive agents” also refer to artificially synthesized materials, molecules or compounds that have a biological effect on a biological cell, tissue or organ. The molecular weights of the bioactive agent can vary from very low (e.g. small molecules, 200-500 Daltons) to very high (e.g. plasmid DNA, ˜2,000,000 Daltons).

Suitable growth factors and cytokines include, but are not limited, to stem cell factor (SCF), granulocyte-colony stimulating factor (G-CSF), granulocyte-macrophage stimulating factor (GM-CSF), stromal cell-derived factor-1, steel factor, VEGF, TGFβ, platelet derived growth factor (PDGF), angiopoeitins (Mg), epidermal growth factor (EGF), bFGF, HNF, NGF, bone morphogenic protein (BMP), fibroblast growth factor (FGF), hepatocye growth factor, insulin-like growth factor (IGF-1), interleukin IL-1α, IL-1β, IL-6, IL-7, IL-8, IL-11, and IL-13, colony-stimulating factors, thrombopoietin, erythropoietin, fit3-ligand, and tumor necrosis factor α (TNFα). Other examples are described in Dijke et al., “Growth Factors for Wound Healing”, Bio/Technology, 7:793-798 (1989); Mulder G D, Haberer P A, Jeter K F, eds. Clinicians' Pocket Guide to Chronic Wound Repair. 4th ed. Springhouse, PA: Springhouse Corporation; 1998:85; Ziegler T. R., Pierce, G. F., and Herndon, D. N., 1997, International Symposium on Growth Factors and Wound Healing: Basic Science & Potential Clinical Applications (Boston, 1995, Serono Symposia USA), Publisher: Springer Verlag.

In some embodiments, suitable bioactive agents include but not limited to therapeutic agents. As used herein, the term “therapeutic agent” refers to a substance used in the diagnosis, treatment, or prevention of a disease. Any therapeutic agent known to those of ordinary skill in the art to be of benefit in the diagnosis, treatment or prevention of a disease is contemplated as a therapeutic agent in the context of the present invention. Therapeutic agents include pharmaceutically active compounds, hormones, growth factors, enzymes, DNA, plasmid DNA, RNA, siRNA, viruses, proteins, lipids, pro-inflammatory molecules, antibodies, antibiotics, anti-inflammatory agents, anti-sense nucleotides and transforming nucleic acids or combinations thereof. Any of the therapeutic agents may be combined to the extent such combination is biologically compatible.

Exemplary therapeutic agents include, but are not limited to, those found in Harrison's Principles of Internal Medicine, 13^{th} Edition, Eds. T. R. Harrison et al. McGraw-Hill N.Y., N.Y.; Physicians Desk Reference, 50^{th} Edition, 1997, Oradell New Jersey, Medical Economics Co.; Pharmacological Basis of Therapeutics, 8^{th} Edition, Goodman and Gilman, 1990; United States Pharmacopeia, The National Formulary, USP XII NF XVII, 1990; current edition of Goodman and Oilman's The Pharmacological Basis of Therapeutics; and current edition of The Merck Index, the complete contents of all of which are incorporated herein by reference.

Examples of therapeutic agents, include but are not limited to, narcotic analgesic drugs; salts of gold; corticosteroids; hormones; antimalarial drugs; indole derivatives; pharmaceuticals for arthritis treatment; antibiotics, including Tetracyclines, Penicillin, Streptomycin and Aureomycin; antihelmintic and canine distemper drugs, applied to domestic animals and large cattle, such, as, for example, phenothiazine; drugs based on sulfur, such, as sulfioxazole; antitumor drugs; pharmaceuticals supervising addictions, such as agents controlling alcohol addiction and agents controlling tobacco addiction; antagonists of drug addiction, such, as methadone; weightcontrolling drugs; thyroid gland controlling drugs; analgesics; drugs controlling fertilization or contraception hormones; amphetamines; antihypertensive drugs; anti-inflammatory agents; antitussives; sedatives; neuromuscular relaxants; antiepileptic drugs; antidepressants; antidisrhythmic drugs; vasodilating drugs; antihypertensive diuretics; antidiabetic agents; anticoagulants; antituberculous agents; antipsyhotic agents; hormones and peptides. It is understood that above list is not full and simply represents the wide diversification of therapeutic agents that can be used. In some embodiments, therapeutic agent is Mitoxantrone, protein (e.g. VEGF) or plasmid DNA.

The amount of bioactive agent in the polymer matrix depends on various factors including, for example, specific agent; function which it should carry out; required period of time for release of a the agent; quantity to be administered. Generally, dosage of an bioactive agent is selected from the range about from 0.001% (w/w) up to 95% (w/w), preferably, from about 5% (w/w) to about 75% (w/w), and, most preferably, from about 10% (w/w) to about 60% (w/w).

In some embodiments, the composition comprises a cell, e.g. a biological cell. One way to incorporate cells into the composition is by reswelling a dried or partially dried composition of the invention in an aqueous solution comprising the cells to be incorporated. The aqueous solution can comprise from about 10⁴ to about 10⁸ cells/ml. In some embodiments, aqueous solution comprises from about 10⁴ to about 10⁶ cells/ml. In some embodiments, aqueous solution comprises about 10⁵ cells/ml. In one embodiment, aqueous solution comprises about 5×10⁵ cells/ml.

In some embodiments, the composition comprises more than one cell type. This can be accomplished by having two or more different cell types in aqueous solution used for swelling. When two or more different cell types are to be incorporated into the composition, the concentration of cells in the aqueous solution ranges from 10³ to about 10⁹ cells/ml. Preferably, concentration of cells in the aqueous solution is 10⁶-10⁸ cells/ml.

Cells amenable to be incorporated into the composition include, but are not limited to, stem cells (embryonic stem cells, mesenchymal stem cells, bone-marrow derived stem cells and hematopoietic stem cells), chrondrocytes progenitor cells, pancreatic progenitor cells, myoblasts, fibroblasts, keratinocytes, neuronal cells, glial cells, astrocytes, pre-adipocytes, adipocytes, vascular endothelial cells, hair follicular stem cells, endothelial progenitor cells, mesenchymal cells, neural stem cells and smooth muscle progenitor cells.

In some embodiments, the cell is a genetically modified cell. A cell can be genetically modified to express and secrete a desired compound, e.g. a bioactive agent, a growth factor, differentiation factor, cytokines, and the like. Methods of genetically modifying cells for expressing and secreting compounds of interest are known in the art and easily adaptable by one of skill in the art.

Differentiated cells that have been reprogrammed into stem cells can also be used. For example, human skin cells reprogrammed into embryonic stem cells by the transduction of Oct3/4, Sox2, c-Myc and Klf4 (Junying Yu, et. al., 2007, Science 318: 1917-1920; Takahashi K. et. al., 2007, Cell 131: 1-12).

Cells useful for incorporation into the composition can come from any source, for example human, rat or mouse. Human cells include, but are not limited to, human cardiac myocytes-adult (HCMa), human dermal fibroblasts-fetal (HDF-f), human epidermal keratinocytes (HEK), human mesenchymal stem cells-bone marrow, human umbilical mesenchymal stem cells, human hair follicular inner root sheath cells, human umbilical vein endothelial cells (HUVEC), and human umbilical vein smooth muscle cells (HUVSMC), human endothelial progenitor cells, human myoblasts, human capillary endothelial cells, and human neural stem cells.

Exemplary rat and mouse cells include, but not limited to, RN-h (rat neurons-hippocampal), RN-c (rat neurons-cortical), RA (rat astrocytes), rat dorsal root ganglion cells, rat neuroprogenitor cells, mouse embryonic stem cells (mESC) mouse neural precursor cells, mouse pancreatic progenitor cells mouse mesenchymal cells and mouse endodermal cells.

In some embodiments, tissue culture cell lines can be used in the compositions described herein. Examples of cell lines include but are not limited to C166 cells (embryonic day 12 mouse yolk), C6 glioma Cell line, HL1 (cardiac muscle cell line), AML12 (nontransforming hepatocytes), HeLa cells (cervical cancer cell line) and Chinese Hamster Ovary cells (CHO cells).

An ordinary skill artisan in the art can locate, isolate and expand such cells. In addition, the basic principles of cell culture and methods of locating, isolation and expansion and preparing cells for tissue engineering are described in “Culture of Cells for Tissue Engineering” Editor(s): Gordana Vunjak-Novakovic, R. Ian Freshney, 2006 John Wiley & Sons, Inc., and in “Cells for tissue engineering” by Heath C. A. (Trends in Biotechnology, 2000, 18:17-19) and these are hereby incorporated by reference in their entirety.

The bioactive agent can be simply physically entrapped can be simply physically entrapped within the matrix, or it can be chemically bound into the matrix or complexed, encased in, or physically immobilized by an intermediate ligand or linker which is in turn, chemically bound into the matrix.

The bioactive agent can be reversibly bound to the polymer matrix, so release of the bioactive agent from the polymer matrix can be sustained over a period of time, and the release rate being enhanced in a controlled manner with ultrasound. Because bioactive agent release does not involve degradation of the polymer matrix, the release rate is relatively easy to control and the bioactivity of released the released agent is not affected by interactions with degradation products of the cross-linked polymer matrix. The polymer matrices can also be modified to enhance or modify their reversible binding to the bioactive agents. See for example, U.S. Pat. No. 7,186,413, contents of which are herein incorporated in their entirety.

The bioactive agent can be covalently linked to the matrix through a linker. The linker can be a cleavable linker or non-cleavable linker, depending on the application. As used herein, a “cleavable linker” refers to linkers that are capable of cleavage under various conditions. Conditions suitable for cleavage can include, but are not limited to, pH, UV irradiation, enzymatic activity, temperature, hydrolysis, elimination and substitution reactions, redox reactions, and thermodynamic properties of the linkage. In many cases, the intended nature of the conjugation or coupling interaction, or the desired biological effect, will determine the choice of linker group. In some embodiments, the bioactive agent is linked to the polymer matrix by a cleavable linker. In some embodiments, the cleavable linker has in vivo half-life of 1 hour, 2 hours, 3 hours, 5 hours, 8 hours, 12 hours, 24 hours, 2 days, 3, days, 4, days, 5 days, 6 days, 7 days, 2 weeks, 3 weeks, 4 weeks, 2 months, 3 months, 6 months or 1 year or more.

In some embodiments, the bioactive agent is bound to the matrix by a hydrolyzable bond. The term “hydrolyzable” refers to bonds which are hydrolyzed or cleaved under physiological conditions. In some embodiments, the hydrolyzable bond is cleaved under the conditions present in serum or blood of a subject. In some embodiments, the hydrolyzable bond has in vivo half-life of 1 hour, 2 hours, 3 hours, 5 hours, 8 hours, 12 hours, 24 hours, 2 days, 3, days, 4, days, 5 days, 6 days, 7 days, 2 weeks, 3 weeks, 4 weeks, 2 months, 3 months, 6 months or 1 year or more.

In some embodiments, the bioactive agent is reversibly linked to the polymer matrix by ionic interactions.

The incorporation of the bioactive agent within the matrix can be achieved in several ways. For example, in one method dry or incompletely swollen matrix may be swelled in an appropriate solution comprising the bioactive material. According to another method the matrix is prepared by a process involving a cross-linking reaction carried out in a medium comprising the bioactive agent. The bioactive agent can also be adsorbed to the surface of the matrix using a variety of secondary interactions (e.g., charge) or covalently coupled to the surface or bulk of the matrix via a degradable or hydrolyzable bond either before or after cross-linking.

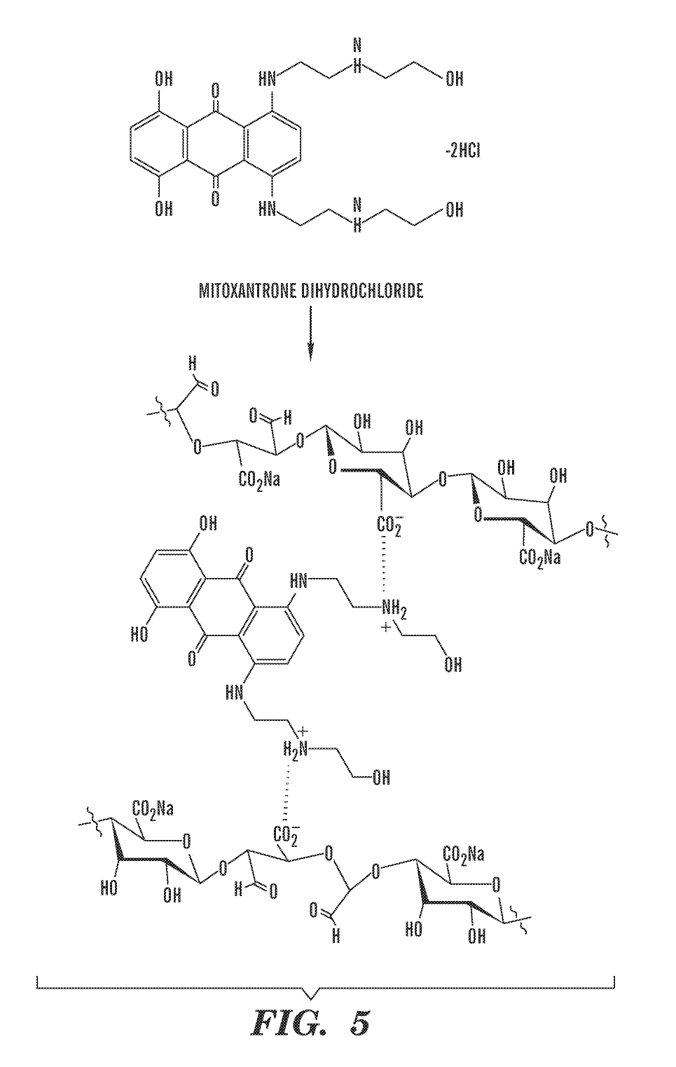

One requirement for controlling release of bioactive agent from the polymer matrix is that the bioactive agent is not readily released from the matrix by molecular diffusion—thus, the mean-free path of the bioactive agent in the polymer matrix must be significantly shorter that the mean-free path in water. Accordingly, in some embodiments, the cell or the bioactive agent has a mean free path in the composition that is shorter than the mean free path of the cell or the bioactive agent in water. When the molecular size of the drug is high enough (e.g. plasmid DNA) that particle size is of similar magnitude to the size of pores in the polymer matrix, this requirement is met, and thus release of the drug will be switched on by external stimulus. The sustained and on-demand release of low-molecular-weight bioactive agents, however, can be achieved by their reversible binding with the polymer matrix. For example, anti-cancer drug Mitoxantrone may form ionic complexes with the carboxylate groups on the sugar residues of the alginate polymer backbone (FIG. 5 and Bouhadir 2001).

The polymer matrix encapsulated bioactive agents can be administered to a subject by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. As used herein, the term “administer” refers to the placement of matrix encapsulated bioactive agent into a subject by a method or route which results in at least partial localization of the bioactive agent at a desired site such that desired effect is produced.

Exemplary modes of administration include, but are not limited to, injection, infusion, instillation, inhalation, or ingestion. “Injection” includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion.

The polymer matrix encapsulated bioactive agent can be delivered to an in vivo locus. Exemplary in vivo loci include, but are not limited to site of a wound, trauma or disease. The matrix encapsulated bioactive agent can be delivered to the in vivo locus by, for example, implanting or injecting the matrix into a subject. The polymer matrix encapsulated bioactive agent can also double as a bulking agent.

Once the polymer matrix is subjected to ultrasonic energy, the acoustic wave causes rapid alternative compressions and tensions of the liquid and solid in the polymer matrix. The alternative compressions and tensions result in cavitations, which enhance the convection of bioactive agent through them out of the matrix. In addition, the collapse of cavitation bubbles can create pronounced perturbation in its surrounding, which can induce the detachment of reversibly bonded bioactive agents on the polymer matrix. As bioactive agents transport through cavitations out of the matrix, physiological fluid containing divalent ions (e.g. Ca²⁺, Mg²⁺, Ba²⁺, and/or Sr²⁺) flows into the cavity. These divalent ions re-cross-link the polymers in the cavities, and dynamically re-heal the polymer matrix. In this way, bioactive agents are released from a polymer matrix at controlled rates upon ultrasound irradiation, without varying the integrity and mechanical strength of the polymer.

Notably, ultrasound has been extensively used to introduce cavitations into skins and cell membranes, and therefore temporally and reversibly enhance its permeability for various bioactive agents. These cavities will soon re-heal, as the ultrasound is withdrawn. See e.g., U.S. Pat. Nos. 4,767,402, 6,002,961, and 4,780,212, contents of all of which are herein incorporated by reference in their entirety. Polymer-matrix systems that release bioactive agents by mimicking these natural, self-healing materials, have the advantage of not being limited to a particular anatomic location, and have the ability to fine-tune the frequency and intensity of ultrasound that will cause release by altering physical aspects of the polymer matrix. (e.g. cross-linking ion type, affinity of the polymers for the cross-linking ions).

The choices of the frequency and intensity depend on the polymer matrix used and the bioactive agents incorporated in the matrix. Representative suitable ultrasonic frequencies are between about 1 kHz and about 1 MHz, while the intensities can range between 0.1 watt and about 30 watts. See, for example, U.S. Pat. No. 4,657,543, contents of which are herein incorporated by reference in their entirety. In some embodiments, ultrasound frequency is from about 20 kHz to 1 MHz. Preferably, ultrasound frequency is about 20 kHZ. In some embodiments, the ultrasound intensity is from about 1 mW to 5 W. Preferably the ultrasound intensity is about 1 W.

The time at which the polymer system is exposed to ultrasound can vary over a wide range depending on the environment. Generally suitable times are between about few seconds and hours. See, for example, U.S. Pat. No. 4,657,543. Notably, these ultrasound power levels are below those which would be considered harmful (Mitragotri 2005). In some embodiments the polymer is exposed to ultrasound for about 1 to about 5 minutes per hour. Preferably, the polymer is exposed to ultrasound for about 5 minutes per hour. The exposure can be in one continuous time period or a pulse system where the total exposure over one hour totals up to the times described above. In one non-limiting example, ultrasound is applied for about 1 to about 15 continuous minutes in a given one hour period. In another non-limiting example, ultrasound is applied in shorter time periods that total up to 1 to 15 minutes in a given one hour period.

For administration to a subject, polymer matrix with the encapsulated bioactive agent can be formulated together with one or more pharmaceutically acceptable carriers (additives) and/or diluents. The pharmaceutical composition can be specially formulated for administration in solid or liquid form, including those adapted for the following: (1) oral administration, for example, drenches (aqueous or non-aqueous solutions or suspensions), lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; (8) transmucosally; or (9) nasally.

Matrices that are to be implanted can additionally include one or more additives. Additives may be resolving (biodegradable) polymers, mannitol, starch sugar, inosite, sorbitol, glucose, lactose, saccharose, sodium chloride, calcium chloride, amino acids, magnesium chloride, citric acid, acetic acid, hydroxyl-butanedioic acid, phosphoric acid, glucuronic acid, gluconic acid, poly-sorbitol, sodium acetate, sodium citrate, sodium phosphate, zinc stearate, aluminium stearate, magnesium stearate, sodium carbonate, sodium bicarbonate, sodium hydroxide, polyvinylpyrolidones, polyethylene glycols, carboxymethyl celluloses, methyl celluloses, starch or their mixtures.

Implants can be formed as a slab which can be circular, rectangular or the like and having a thickness between 0.1 mm and about 100 mm in a total surface area between about 0.01 cm² and about 100 cm².

Implant can be of cylindrical form from about 0.5 to about 10 mm in diameter and from about 0.5 to about 10 cm in length. Preferably, its diameter is from about 1 to about 5 mm and length from about 1 to about 5 cm. In some cases, implant can be of spherical form.

When the implant is in a spherical form, its diameter can range from about 0.5 to about 50 mm in diameter. In some embodiments, a spherical implant's diameter is from about 5 to about 30 mm. Preferably the diameter is from about 10 to about 25 mm.

In some embodiments, implants can comprise multiple particles of the self-healing polymer. Said particles can range in size from about 10 nm to about 500 micros in size. Without wishing to be bound by theory, implants comprising multiple particles can take any shape and not limited to regular shapes described above. For example, the particles can be arranged in shapes that are irregular, i.e., shapes that do not have a defined geometry.

### Controlled Drug Release

For bioactive agents that are immobilized in the matrix, a controlled release rate is achieved under ultrasound irradiation. When ultrasound is off, the release stops, due to the re-healing of the matrix. Therefore, release of the bioactive agent can be achieved in a pulsalite manner by repeated application of external stimulus.

This ability for on-demand pulsatile release of bioactive agents is useful in a variety of settings, including immunizations, which typically provide an initial immunization, followed by distinct booster doses at later times. For bioactive agents that can naturally diffuse out of the matrix, a baseline release rate exists without external stimulus. However, ultrasound irradiation can enhance the release rate to multiple times of the baseline value. This baseline-enhanced release profile is useful in many contexts, including increasing release of pain killers for short times to deal with acute more intense pain. Moreover, repeated administration of well-defined doses of allergen, in the absence of immunostimulatory molecules, from such a polymer matrix may be useful for inducing tolerance. It may also be advantageous to use an external stimulus to deliver bioactive agents in a pulsatile manner so as to time the delivery to coincide with a particular biological event (e.g. the circadian rhythm) in order to maximize the effectiveness of the bioactive agent (e.g. against tumors).

In other cases, the polymer matrix can be designed to continuously release one bioactive agent, and on-demand delivery of antibodies or other macromolecules that enhance or decrease the bioactivity of this bioactive agent may be used as a remotely activated “on-switch or off-switch,” to allow the activity of the bioactive agent to be increased or decreased in emergent clinical situations (e.g. the need to hold chemotherapeutic bioactive agents with immunodepressant side-effects while treating infections) without removing the bioactive agent-eluting device. Similarly, the system may be designed to continuously release one bioactive agent that initiates a desirable biological process, with release of a second or third bioactive agent triggered on demand at the appropriate stage of this process. There are many examples of the need for sequentially acting morphogens and growth factors in the tissue engineering and regeneration fields. For example, it would be desirable to provide continuous release of a first bioactive agent (e.g., vascular endothelial growth factor) that activates angiogenesis to treat ischemic diseases, with triggered release of a second bioactive agent (e.g., platelet derived growth factor) to drive maturation of the newly formed vessels once a sufficient density has been formed via the action of the first molecule.

It is to be understood that under the same conditions a released bioactive agent has bioactivity that is comparable to that when the bioactive agent was not first encapsulated in the polymer matrix. In some embodiments, the released bioactive agent has bioactivity that is at least 50%, 60%, 70%, 80%, 90%, 95% or 100% of the bioactivity when the agent was not first encapsulated by the polymer matrix.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

Other than in the operating examples, or where otherwise indicated, all numbers expressing quantities of ingredients or reaction conditions used herein should be understood as modified in all instances by the term “about.” The term “about” when used in connection with percentages may mean ±1%. Furthermore, the term “about” can mean within ±1% of a value.

The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description.

Although methods and materials similar or equivalent to those described herein can be used in the practice or testing of this disclosure, suitable methods and materials are described below. The term “comprises” means “includes.” The abbreviation, “e.g.” is derived from the Latin exempli gratia, and is used herein to indicate a non-limiting example. Thus, the abbreviation “e.g.” is synonymous with the term “for example.”

The terms “decrease”, “reduced”, “reduction”, “decrease” or “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g. absent level as compared to a reference sample), or any decrease between 10-100% as compared to a reference level.

The terms “increased”,“increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two standard deviation (2SD) above or below a reference level. The term refers to statistical evidence that there is a difference. It is defined as the probability of making a decision to reject the null hypothesis when the null hypothesis is actually true. The decision is often made using the p-value.

As used here, the term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water, (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

As used herein, the term “polymer” is intended to include both oligomeric and polymeric species, i.e., compounds which include two or more monomeric units, which may be a homopolymer or a copolymer. The term “homopolymer” is a polymer incorporating a single species of monomer units. The term “copolymer” is a polymer constructed from two or more chemically distinct species of monomer units in the same polymer chain. A “block copolymer” is a polymer which incorporates two or more segments of two or more distinct species of homopolymers or copolymers.

As used herein, the term “swelling agent” refers to those compounds or substances which affect at least a degree of swelling. Typically, swelling agents is an aqueous solution or organic solvent, however swelling agent can also be a gas. In some embodiments, swelling agent is water or a physiological solution, e.g. phosphate buffer saline, or growth media

As used herein, the term “linker” means an organic moiety that connects two parts of a compound. Linkers typically comprise a direct bond or an atom such as oxygen or sulfur, a unit such as SS, NH, C(O), C(O)NH, SO, SO₂, SO₂NH or a chain of atoms, such as substituted or unsubstituted alkyl where one or more methylenes can be interrupted or terminated by O, S, S(O), SO₂, NH, NH₂, C(O).

By “treatment”, “prevention” or “amelioration” of a disease or disorder is meant delaying or preventing the onset of such a disease or disorder, reversing, alleviating, ameliorating, inhibiting, slowing down or stopping the progression, aggravation or deterioration the progression or severity of a condition associated with such a disease or disorder. In one embodiment, the symptoms of a disease or disorder are alleviated by at least 5%, at least 10%, at least 20%, at least 30%, at least 40%, or at least 50%.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments, the subject is a mammal, e.g., a primate, e.g., a human. The terms, “patient” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used as subjects that represent animal models of HIF or hypoxia related pathologies. In addition, the methods described herein can be used to treat domesticated animals and/or pets. A subject can be male or female. A subject can be one who has been previously diagnosed with or identified as suffering from or having a HIF or hypoxia related pathology, one or more complications related to a HIF or hypoxia related pathology, and optionally, but need not have already undergone treatment for such a HIF or hypoxia related pathology.

All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

To the extent not already indicated, it will be understood by those of ordinary skill in the art that any one of the various embodiments herein described and illustrated may be further modified to incorporate features shown in any of the other embodiments disclosed herein.

The present invention may be defined in any of the following numbered paragraphs:
1. A method for releasing a bioactive agent on demand in response to ultrasound, the method comprising providing a physiologically acceptable self-healing polymer matrix comprising the bioactive agent, and inducing cavitation in the polymer matrix via ultrasound to release the bioactive agent, wherein the self-healing polymer matrix is reversibly cross-linked.
2. The method of paragraph 1, wherein the polymer matrix is reversibly cross-linked via divalent ion cations selected from the group consisting of Ca²⁺, Mg²⁺, Ba²⁺, Sr²⁺, and any combinations thereof.
3. The method of any of paragraphs 1-2, wherein polymer matrix is reversibly cross-linked under physiological conditions.
4. The method of any of paragraphs 1-3, wherein the polymer matrix comprises alginate or derivative(s) thereof.
5. The method of any of paragraphs 1-4, wherein the bioactive agent has a mean free path of diffusion in the polymer matrix that is shorter than the mean free path for diffusion of the same bioactive agent in water.
6. The method of any of paragraphs 1-5, wherein the bioactive agent is reversibly bound to the polymer matrix.
7. The method of any of paragraphs 1-6, wherein the bioactive agent is bound to the polymer matrix by ionic interactions.
8. The method of any of paragraphs 1-6, wherein the bioactive agent is bound to the polymer matrix by a hydrolyzable bond.
9. The method of any of paragraphs 1-6, wherein the bioactive agent is bound to the polymer matrix by a cleavable linker.
10. The method of any of paragraphs 1-9, wherein the ultrasound has a frequency from about 20 KHz to about 1 MHz.
11. The method of any of paragraphs 1-10, wherein the ultrasound has an intensity from about 1 watt to about 30 watts.
12. The method of any of paragraphs 1-11, wherein the polymer matrix has a molecular weight from about 5,000 Daltons to about 500,000 Daltons.
13. The method of any of paragraphs 1-12, wherein the released bioactive agent has a bioactivity that is comparable to that when the bioactive agent was not first encapsulated in the polymer matrix.
14. The method of any of paragraphs 1-13, wherein the polymer matrix has physical integrity and/or mechanical stiffness values that are within 24%, 10%, 5%, 2% or less of their initial values after repeated applications of ultrasound.
15. The method of any of paragraphs 1-14, wherein the polymer matrix is biodegradable.
16. The method according to any of paragraphs 1-15, further comprising providing the polymer matrix to a location within a subject.

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLES

### Example 1

### In Vitro Release of Encapsulated Mitoxantrone

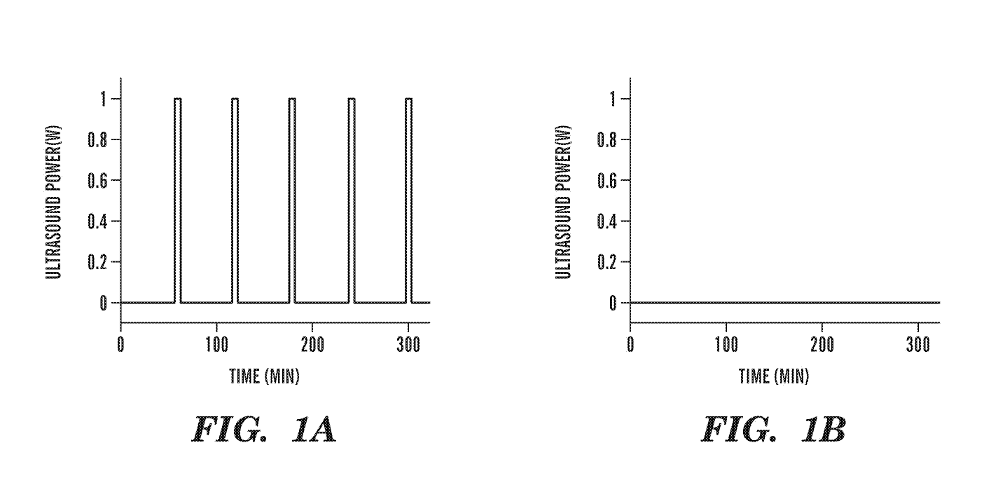

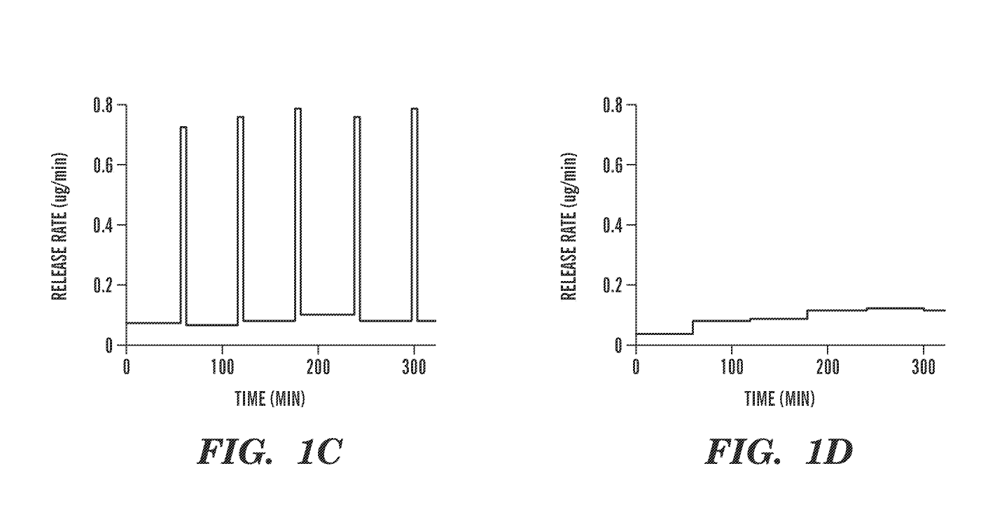

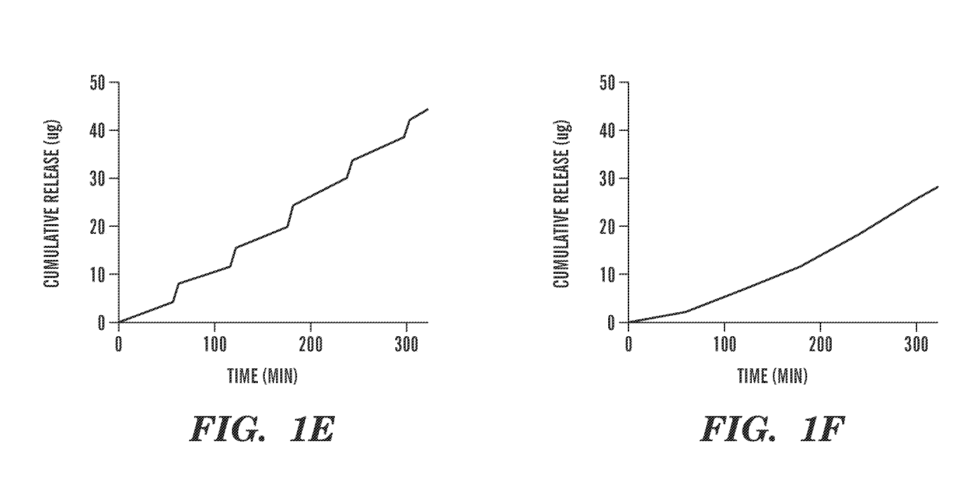

Alginate hydrogels containing 0.825 mg/mL of Mitoxantrone were prepared by mixing alginate aqueous solution with slurries of CaSO₄, to a final concentration of 50 mM Ca² and 2% alginate (w/w) polymer. The gel was cast between two glass slides and cut into discs of approximately 10 mm diameter and 2 mm thickness. Thereafter, hydrogels were swollen overnight in the commercially available Dulbecco's Modified Eagle's Medium (DMEM). Hydrogel disks so prepared were next placed in a 15 mL plastic tube with 5 ml of DMEM. One group of the sample hydrogels were subjected to low-frequency ultrasound with frequency of 20 KHz and intensity of 1 watt for 5 min per hour. A further group comprising control hydrogels, similarly prepared, were not subjected to any stimulation. FIGS. 1A and 1B show the power profiles of ultrasound on the two groups of hydrogels. As seen from FIG. 1C, ultrasound stimulation increased the release rates of Mitoxantrone by ˜10 times, and the enhanced release rates were all on the same level. This showed that the release of Mitoxantrone was on-demand and controlled. As seen from comparison of FIGS. 1D and 1E, just 5 min of ultrasound per hour increased the cumulative release amount by ˜70%. Moreover, the control of drug release in the matrix was nearly digital, and the rate of Mitroxantrone release decayed to negligible levels soon after removing the ultrasound.

### Example 2

### Bioactivity of Encapsulated Mitoxantrone after Ultrasound Release from Hydrogel

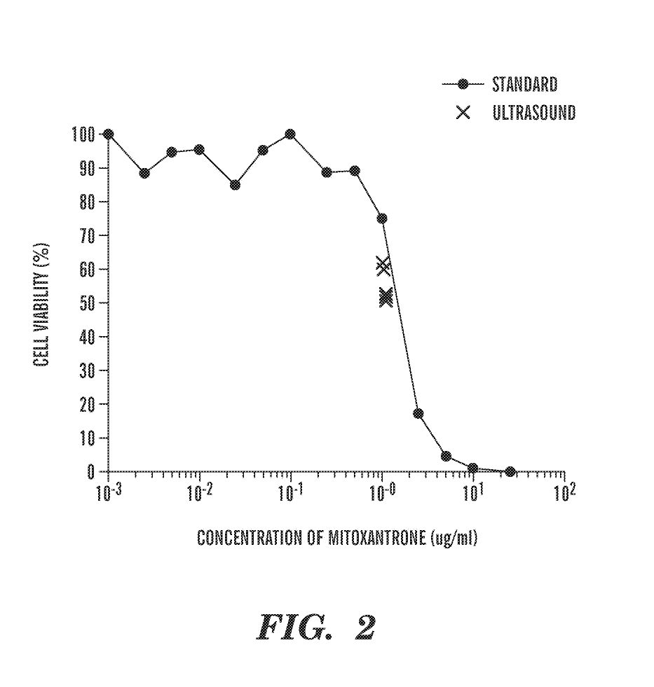

The bioactivity of the released Mitoxantrone in example 1 was studied by incubating MCF7 breast cancer cells in the Mitoxantrone solution, and measuring the cell viability after 24 hours. Fresh Mitoxantrone solutions (unmixed with polymer) with various concentrations ranging from 0.1 to 20 μg/mL were also used to incubate the same number of MCF7 breast cancer cells. From FIG. 2. it can be seen that the ultrasound-released Mitoxantrone reduces cell viability by the same amount as a fresh Mitoxantrone solution of equivalent concentration. Therefore, ultrasound-induced drug delivery can maintain the bioactivity of the released drug.

### Example 3

### In Vitro Release of Encapsulated Plasmid DNA

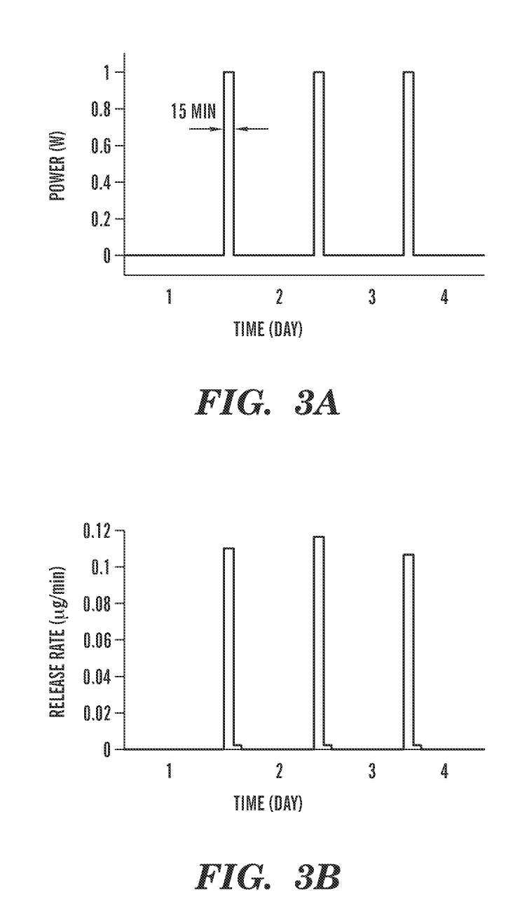

Alginate hydrogels containing 0.1 mg of plasmid DNA per 1 ml of the gel were prepared as described in Example 1. After swelling in DMEM, the hydrogel disks were subjected to low-frequency ultrasound with frequency of 20 KHz and intensity of 1 watt for 15 min each day. FIG. 3A shows the power profiles of ultrasound on the hydrogel. FIG. 3B shows that ultrasound increased the release rate from almost 0 to a finite value, and the release rates with ultrasound were similar over three days. This again shows that release of Plasmid DNA is on-demand and controlled.

### Example 4

### Properties of Polymer Matrix under Ultrasound in Various Environments

Alginate hydrogels prepared as in Example 1 and swollen in DMEM were placed in plastic tube with 5 ml of regular DMEM (with 200 mg/L of CaCl₂ and 100 mg/L of MgSO₄), modified DMEM (with 1000 mg/L of CaCl₂ and 500 mg/L of MgSO₄), or PBS (without Ca²⁺ and Mg²⁺). Low-frequency ultrasound with frequency of 20 kHz and intensity of 1 watt was applied to the hydrogel disks in various solutions for 10 min each hour. The elastic modulus of the gel disks were measured after each ultrasound irradiation. The hydrogel disks were then lyophilized to measure their dry weights.

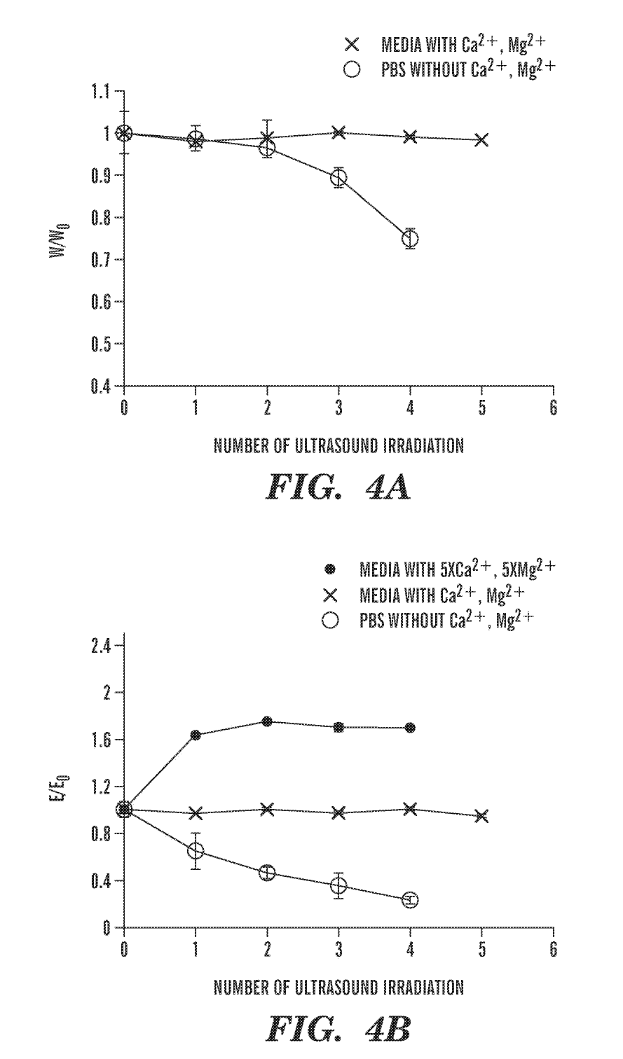

FIG. 4A shows that the measured elastic modulus of gel disks in PBS reduced by ˜80% after four times of ultrasound irradiation. On the other hand, the elastic modulus of gel disks in modified DMEM increased by ˜80% after one ultrasound irradiation, and maintained at the same level with further irradiations. The elastic modulus of gel disks in regular DMEM kept almost the same under ultrasound irradiations. FIG. 4B shows that hydrogel disks in PBS lost ˜30% of their weights after four times of ultrasound irradiation, but the hydrogel disks in regular DMEM maintained almost constant weight. Hydrogels in regular DMEM were found to be still intact after ultrasound irradiations, but the hydrogel disk irradiated in PBS had been severely damaged with cavities generated in it (data not shown).

The concentration of Ca²⁺ and Mg²⁺ in physiological environment is close to the concentration in regular DMEM. The above results show that the Ca²⁺ and Mg²⁺ in physiological environment are sufficient to re-heal the cavities caused the low frequency-ultrasound, and therefore maintain the physical integrity and mechanical modulus of gels subjected to repeated ultrasound.

### REFERENCES

1. Bouhadir, K. H. (2001). “Hydrogels for combination delivery of antineoplastic agents.” Biomaterials 22(19): 2625-2633.
2. Cheng T. J. (2006). “Magnetically responsive polymeric microparticles for oral delivery of protein drugs.” Pharm Res 23(3): 557-64.
3. Mitragotri S (2005). “Healing sound: the use of ultrasound in drug delivery and other therapeutic applications.” Nat Rev Drug Disc 4(3): 255-60.
4. Langer, R. (1990). “New Methods of Drug Delivery” Science 249(4976): 1527-1533.
5. Langer, R. (1998). “Drug delivery and targeting.” Nature 392(6679): 5-10.
6. Lee, K. Y. (2001). “Hydrogels for tissue engineering.” Chemical Reviews 101(7): 1869-1879.
7. Richardson, T. P. (2001). “Polymeric system for dual growth factor delivery.” Nature Biotechnology 19(11): 1029-1034.
8. Thornton A J. (2004). “Shape retaining injectable hydrogels for minimally invasive bulking.” J Urol. 127(2): 763-8.
9. Uhrich, K. E. (1999). “Polymeric systems for controlled drug release.” Chemical Reviews 99(11): 3181-3198.

What is claimed is:

1. A method for releasing a bioactive agent on demand in response to ultrasound, the method comprising providing a physiologically acceptable self-healing polymer matrix comprising the bioactive agent, and inducing cavitation in the polymer matrix via ultrasound to release the bioactive agent, wherein the self-healing polymer matrix is reversibly cross-linked.

2. The method of claim 1, wherein the polymer matrix is reversibly cross-linked via divalent ion cations selected from the group consisting of Ca²⁺, Mg²⁺, Ba²⁺, Sr²⁺, and any combinations thereof.

3. The method of claim 1, wherein polymer matrix is reversibly cross-linked under physiological conditions.

4. The method of claim 1, wherein the polymer matrix comprises alginate or derivative(s) thereof.

5. The method of claim 1, wherein the bioactive agent has a mean free path of diffusion in the polymer matrix that is shorter than the mean free path for diffusion of the same bioactive agent in water.

6. The method of claim 1, wherein the bioactive agent is reversibly bound to the polymer matrix.

7. The method of claim 6, wherein the bioactive agent is bound to the polymer matrix by ionic interactions.

8. The method of claim 6, wherein the bioactive agent is bound to the polymer matrix by a hydrolyzable bond.

9. The method of claim 6, wherein the bioactive agent is bound to the polymer matrix by a cleavable linker.

10. The method of claim 1, wherein the ultrasound has a frequency from about 20 KHz to about 1 MHz.

11. The method of claim 1, wherein the ultrasound has an intensity from about 1 watt to about 30 watts.

12. The method of claim 1, wherein the polymer matrix has a molecular weight from about 5,000 Daltons to about 500,000 Daltons.

13. The method of claim 1, wherein the released bioactive agent has a bioactivity that is comparable to that when the bioactive agent was not first encapsulated in the polymer matrix.

14. The method of claim 1, wherein the polymer matrix has physical integrity and/or mechanical stiffness values that are within 24%, 10%, 5%, 2% or less of their initial values after repeated applications of ultrasound.

15. The method of claim 1, wherein the polymer matrix is biodegradable.

16. The method of claim 1, further comprising providing the polymer matrix to a location within a subject.

17. The method of claim 1, wherein the polymer matrix is exposed to ultrasound for about 1 to about 5 minutes per hour.

18. The method of claim 1, wherein the polymer matrix is a hydrogel.

19. The method of claim 1, wherein the bioactive agent is has a molecular weight between about 200 Daltons to about 2,000,000 Daltons.

20. The method of claim 1, wherein the bioactive agent is a therapeutic agent.
